(19) United States
(12) Patent Application Publication
(10) Pub. No.: US 2026/0256769 A1
(43) Pub. Date: Sep. 3, 2026

(54) ARTERIOSCLEROSIS SUPPRESSION THROUGH INHIBITION OF TRANSCRIPTION FACTOR FoxO1

(71) Applicant: TEIKYO UNIVERSITY, Tokyo (JP)

(72) Inventors: Daisuke TSUKUI, Tokyo (JP); Hajime KONO, Tokyo (JP); Yoshitaka KIMURA, Tokyo (JP)

(73) Assignee: TEIKYO UNIVERSITY, Tokyo (JP)

(21) Appl. No.: 18/993,180

(22) PCT Filed: Jul. 12, 2023
(86) PCT No.: PCT/JP2023/025687

§ 371 Date: Jan. 10, 2025

(30) Foreign Application Priority Data
Jul. 12, 2022 (JP) 2022-111955

(51) Int. Cl. A61K 31/47; A61K 31/472; A61P 9/10; C07K 16/18; C12N 15/113

(52) U.S. Cl. CPC A61K 31/47; A61K 31/472; A61P 9/10; C07K 16/18; C12N 15/113; C12N 2310/14

(57) ABSTRACT

Provided is a novel pharmaceutical composition for preventing or treating atherosclerosis. The pharmaceutical composition contains a FoXO1 inhibitor as an active ingredient.

### TECHNICAL FIELD

The present invention relates to the suppression of atherosclerosis by inhibiting the transcription factor FoxO1, and more specifically, to a pharmaceutical composition for preventing or treating atherosclerosis containing a FoxO1 inhibitor.

### BACKGROUND ART

In the search for new therapeutic targets for atherosclerosis, the inventors focused on the non-receptor tyrosine kinase Syk (spleen tyrosine kinase) gene, which is highly expressed in human atherosclerosis lesions, and created Syk gene-deficient mice and induced atherosclerosis by feeding them a high-fat diet. As a result, the inventors confirmed that in addition to suppressing atherosclerosis (reducing atherosclerosis lesions), there was also a decrease in macrophages, which play a central role in the formation of atherosclerosis lesions, and a decrease in the cell motility and migration ability of macrophages. In addition, the inventors performed RNA-Seq using bone marrow monocytes from Syk gene-deficient mice, and focused on the adhesion factor CD11c among the genes with reduced expression. The inventors confirmed that CD11c expression was also reduced on the cell surface of peripheral blood monocytes from Syk gene-deficient mice.

In this connection with this, it is known that CD11c is involved in cell migration, and atherosclerosis is suppressed in CD11c knockout mice (Non-patent literature 1).

However, the factors downstream of Syk that regulate CD11c expression have not yet been elucidated.

On the other hand, FoxO1, along with its isoforms FoxO3a and FoxO4, is a transcription factor that belongs to the O subfamily of transcription factors having a forkhead domain (Forkhead bOX-containing proteins, Q subfamily), and is known to be involved in insulin signaling, lipid metabolism, and cell differentiation and proliferation (Non-patent literature 2).

Regarding the relationship between FoxO1 and atherosclerosis, the following reports have been made:
Nuclear overexpression of FoxO1 promotes atherosclerosis (2012, Non-patent literature 3).
Skeletal muscle-specific overexpression of FoxO1 suppresses atherosclerosis (2012, Non-patent literature 4).
Endothelial-specific triple knockout mice of FoxO1/FoxO3a/FoxO4 suppresses atherosclerosis (2012, Non-patent literature 5).
Bone marrow-specific triple knockout mice of FoxO1/FoxO3a/FoxO4 promotes atherosclerosis (2013, Non-patent literature 6).

For reference, Non-patent literatures 3, 5, and 6 are reports from the same group.

Although all reports have confirmed changes in atherosclerosis lesions in mice, completely opposite results have been reported depending on the tissue, and the effect of systemic FoxO1 inhibition on atherosclerosis remains unclear. For example, nuclear overexpression of FoxO1 promotes atherosclerosis (Non-patent literature 3), whereas skeletal muscle-specific overexpression of FoxO1 suppresses atherosclerosis (Non-patent literature 4). Alternatively, endothelial-specific triple knockout mice of FoxO1/FoxO3a/FoxO4 suppresses atherosclerosis (Non-patent literature 5), whereas bone marrow-specific triple knockout mice of FoxO1/FoxO3a/FoxO4 promotes atherosclerosis (Non-patent literature 6).

### CITATION LIST

### Non-Patent Literature

[Non-patent literature 1] Wu H, et al. Functional role of CD11c+ monocytes in atherogenesis associated with hypercholesterolemia. Circulation. 2009; 119 (20): 2708-2717.
[Non-patent literature 2] Adipose Tissue and FoxO1: Bridging Physiology and Mechanisms Cells. 2020 Mar. 31; 9 (4): 849. doi: 10.3390/cells9040849
[Non-patent literature 3] L., Qiang et al, THE JOURNAL OF BIOLOGICAL CHEMISTRY, VOL. 287, NO. 17, pp. 13944-13951, Apr. 20, 2012
[Non-patent literature 4] Y. Shimba, Biochemical and Biophysical Research Communications 540 (2021) 61-66
[Non-patent literature 5] K. Tsuchiya, Cell Metabolism 15, 372-381, Mar. 7, 2012
[Non-patent literature 6] K. Tsuchiya, Circulation Research Mar. 29, 2013, 992-1003

### SUMMARY OF INVENTION

### Technical Problem

Under these circumstances, the inventors aimed to identify the factors that regulate CD11c expression downstream of Syk by using a reporter assay with bone marrow-derived macrophages to explore the CD11c promoter region that is affected by Syk. The inventors found that the CD11c promoter region affected by the target Syk is located approximately 1 kbp upstream from the transcription start site of CD11c. The inventors predicted the transcription factor that binds to the nucleotide sequence in that region, administered an inhibitor of the predicted transcription factor to bone marrow-derived macrophages, and measured CD11c on the cell surface by flow cytometry. The inventors confirmed that administration of the transcription factor FoxO1 inhibitor reduced CD11c expression. Next, when the FoxO1 inhibitor was intraperitoneally administered to atherosclerosis model mice for two weeks, a decrease in CD11c was confirmed in both bone marrow monocytes and peripheral blood monocytes. Therefore, when the FoxO1 inhibitor was intraperitoneally administered to atherosclerosis model mice for eight weeks and atherosclerosis lesions in the aortic sinus were evaluated, suppression of atherosclerosis was confirmed.

Thus, although it was publicly known that atherosclerosis was suppressed in Syk gene-deficient mice and CD11c gene-deficient mice, the existence of factors mediating between Syk and CD11c was unknown at the time of filing the application. Under these circumstances, the inventors discovered that the factor regulating CD11c expression downstream of Syk is the transcription factor FoxO1, and further discovered that when a FoxO1 inhibitor was administered to atherosclerosis model mice, atherosclerosis was suppressed.

The present invention is based on such novel findings, and the problem to be solved by the present invention is to provide a novel pharmaceutical composition for preventing or treating atherosclerosis.

### Solution to Problem

The object can be solved by the pharmaceutical composition of preventing or treating atherosclerosis, comprising a FoxO1 inhibitor as an active ingredient.

### Advantageous Effects of Invention

According to the present invention, a novel pharmaceutical composition for preventing or treating atherosclerosis can be provided. According to the present invention, FoxO1 inhibition may be a novel therapeutic intervention point for atherosclerosis.

### BRIEF DESCRIPTION OF THE DRAWINGS

FIG. 1 is a graph showing the results of a reporter assay to identify the CD11c promoter region affected by Syk.

FIG. 2 is a graph showing the results of administering the FoxO1 inhibitor AS1842856 to bone marrow-derived macrophages respectively collected from Syk gene-deficient mice (Syk^{del/del}) and wild-type mice (Syk^{+/+}), and measuring CD11c on the cell surface by flow cytometry.

FIG. 3 is an image showing the results of Western blotting to evaluate the nuclear distribution of FoxO1 in bone marrow-derived macrophages respectively collected from Syk-deficient mice (Syk^{del/del}) and wild-type mice (Syk^{+/+}).

FIG. 4 is a graph showing the results of measuring CD11c expression by real-time PCR in bone marrow monocytes collected from Syk gene-deficient mice (Syk^{del/del}) and wild-type mice (Syk^{+/+}) after intraperitoneal administration of the FoxO1 inhibitor AS1842856 (20 mg/kg) to the Syk gene-deficient mice (Syk^{del/del}) and the wild-type mice (Syk^{+/+}) for two weeks.

FIG. 5 is a graph showing the results of measuring CD11c expression (%) by flow cytometry in peripheral blood monocytes (CD115-positive cells) collected from Syk gene-deficient mice (Syk^{del/del}) and wild-type mice (Syk^{+/+}) after intraperitoneal administration of the FoxO1 inhibitor AS1842856 (20 mg/kg) to the Syk gene-deficient mice (Syk^{del/del}) and the wild-type mice (Syk^{+/+}) for two weeks.

FIG. 6 is a photograph instead of a drawing showing the results of evaluating atherosclerosis lesions in the aortic sinus by HE staining after intraperitoneal administration of the FoxO1 inhibitor AS1842856 (20 mg/kg) to wild-type mice (Syk^{+/+}) for 8 weeks.

FIG. 7 is a graph showing the results of evaluating atherosclerosis lesions in the aortic sinus by HE staining after intraperitoneal administration of the FoxO1 inhibitor AS1842856 (20 mg/kg) to wild-type mice (Syk^{+/+}) for 8 weeks.

### DESCRIPTION OF EMBODIMENTS

The pharmaceutical composition for preventing or treating atherosclerosis (hereinafter sometimes referred to as the pharmaceutical composition of the present invention), comprising a FoXO1 inhibitor as an active ingredient. FoxO1, along with its isoforms FoxO3a and FoxO4, is a transcription factor that belongs to the O subfamily of transcription factors having a forkhead domain (Forkhead bOX-containing proteins, O subfamily), and is known to be involved in insulin signaling, lipid metabolism, and cell differentiation and proliferation.

Examples of FoxO1 inhibitors include AS1842856 [5-amino-7-(cyclohexylamino)-1-ethyl-6-fluoro-4-oxo-1,4-dihydroquinoline-3-carboxylic acid] represented by the following structural formula:

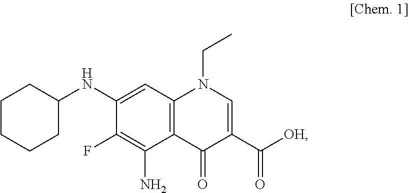 [SMILES: CCn1cc(C(=O)O)c(=O)c2c(N)c(F)c(NC3CCCCC3)cc21]

AS1708727 [2-Cyclopentyl-N-[2,4-dichloro-3-(isoquinolin-5-yloxymethyl)phenyl] N-methylacetamide] represented by the following structural formula:

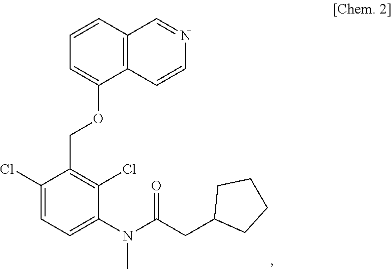 [SMILES: CN(C(=O)CC1CCCC1)c1ccc(Cl)c(COc2cccc3cnccc23)c1Cl]

interfering nucleic acid molecules (for example, siRNA), and anti-FoxO1 antibodies.

Whether a compound is a FoxO1 inhibitor can be evaluated, for example, using mass spectrometry and reporter cells. Specifically, FoxO1 is reacted with a certain compound, and the resulting complex is extracted and dissociated, and then analyzed by a mass spectrometry to identify the compound that binds to FoxO1. Next, confirmation can be made by administering it to reporter cells that have been transfected with a plasmid containing a promoter region known to bind FoxO1 and a reporter gene, and measuring whether or not the transcriptional activity of FoxO1 is suppressed.

These active ingredients can be used alone or in combination of two or more.

The pharmaceutical composition of the present invention can be administered as a medicine to a subject (for example, an animal, preferably a mammal, particularly a human) in an effective amount as an active ingredient alone or, preferably, together with a pharmaceutically acceptable carrier or diluent, or can be provided in the form of a food or drink.

When the pharmaceutical composition of the present invention is provided as a medicine, it can be administered as an oral or parenteral preparation, and is preferably an oral preparation. Examples of the oral preparations include liquid preparations such as a suspension, an emulsion, a syrup, and an extract, and solid preparations such as a tablet, a capsule, a powder, a fine granule, and a granule. Examples of the parenteral preparations include, for example, an injection.

Examples of the pharmaceutically acceptable carriers or diluents include a filler, a disintegrant, a binder, a taste masking agent, a foaming agent, a sweetener, a perfume, a lubricant, a buffer, an antioxidant, a surfactant, and a fluidizer.

The dosage of the pharmaceutical composition of the present invention can be appropriately selected depending on, for example, the condition, body weight, age, and sex of the patient, or the route of administration. In general, the lower limit of the dosage can be selected within the range of about 1 μg to 10 μg per day for an adult, and the upper limit of the dosage can be selected within the range of about 100 μg to 1000 μg per day for an adult, and the composition can be administered in 1 to 3 divided doses per day.

The pharmaceutical composition of the present invention can be used to prevent or treat arteriosclerosis, particularly to inhibit arteriosclerosis.

### EXAMPLES

The present invention will now be further illustrated by, but is by no means limited to, the following examples.

### Example 1: Identification of a Factor that Regulates CD11c Expression Downstream of Syk

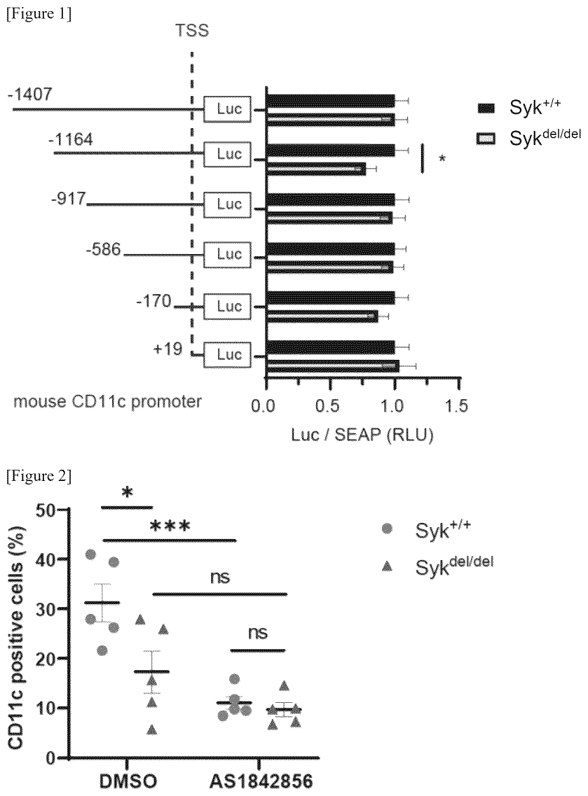

In this Example, the following experiment was carried out. Lentiviral vectors containing CD11c promoter regions of various base lengths that were gradually and partially deleted from the upstream side and luciferase linked downstream of the regions were constructed. Bone marrow-derived macrophages respectively collected from Syk gene-deficient mice (Syk^{del/del}) and wild-type mice (Syk^{+/+}) were infected with the lentiviral vectors. Using the difference in luciferase activity between both mice as an indicator to determine the CD11c promoter region associated with Syk. As a result, it was found that Syk was associated with a region approximately 1 kbp upstream from the CD11c transcription start site (FIG. 1).

Next, we assumed that the transcription factor that binds to the nucleotide sequence of the determined region as above was FoxO1, and a FoxO1 inhibitor AS1842856 was administered to the bone marrow-derived macrophages respectively collected from Syk gene-deficient mice (Syk^{del/del}) and wild-type mice (Syk^{+/+}), and CD11c on the cell surface was measured by flow cytometry. In the bone marrow-derived macrophages collected from wild-type mice, the expression of CD11c was significantly decreased in the AS1842856-treated group compared to the control group (dimethyl sulfoxide (DMSO)-treated group) (FIG. 2).

In FIG. 2, as shown in a comparison of the DMSO-treated group of bone marrow-derived macrophages group collected from wild-type mice (Syk^{+/+}) with DMSO-treated group of bone marrow-derived macrophages collected from Syk gene-deficient mice (Syk^{del/del}), in bone marrow-derived macrophages collected from Syk gene-deficient mice, the expression of CD11c was originally reduced, and no significant difference was observed between the control group (DMSO-treated group) and the AS1842856-treated group in Syk gene-deficient mice.

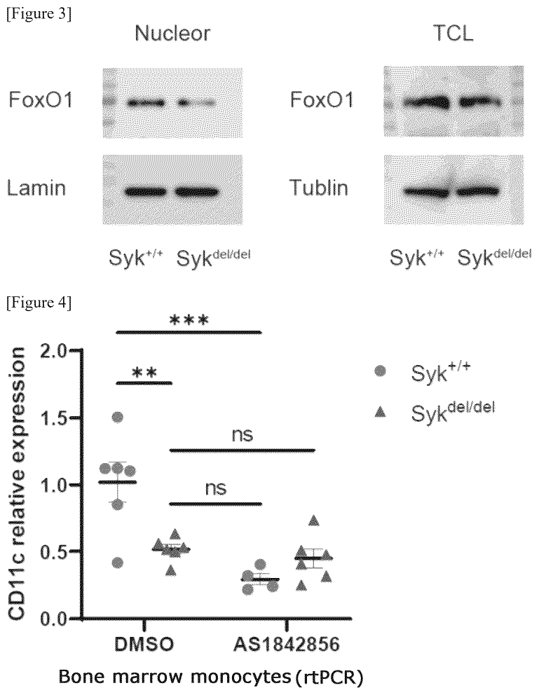

The transcription factor FoxO1 is distributed in the nucleus and cytoplasm, and translocates into the nucleus depending on conditions and stimuli to promote transcription. Therefore, the nuclear distribution of FoxO1 in bone marrow-derived macrophages respectively collected from Syk gene-deficient mice (Syk^{del/del}) and wild-type mice (Syk^{+/+}) was evaluated by Western blotting. In total cell lysate (TCL), no difference in the FoxO1 level was observed between both, but nuclear FoxO1 level was decreased in bone marrow-derived macrophages collected from Syk gene-deficient mice (FIG. 3).

These results indicate that the factor that regulates the expression of CD11c downstream of Syk is the transcription factor FoxO1.

### Example 2: Suppression of Atherosclerosis by Inhibiting FoxO1

In this Example, the following experiment was carried out. First, the FoxO1 inhibitor AS1842856 (20 mg/kg) was intraperitoneally administered to Syk gene-deficient mice (Syk^{del/del}) and wild-type mice (Syk^{+/+}) for 2 weeks. The expression of CD11c in collected bone marrow monocytes was measured by real-time PCR, and the CD11c expression (%) in peripheral blood monocytes (CD115 positive cells) was measured by flow cytometry. CD115 is a monocyte marker.

Regard the CD11c expression in bone marrow monocytes, for bone marrow monocytes collected from wild-type mice, the CD11c expression was significantly decreased in the AS1842856-treated group, compared to the control group (DMSO-treated group) (FIG. 4).

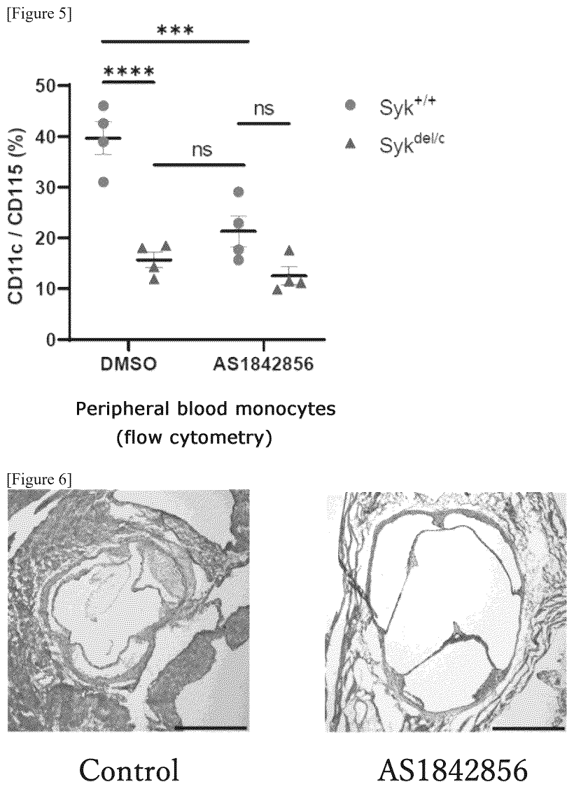

Regarding the CD11c expression (%) in peripheral blood monocytes (CD115 positive cells), for peripheral blood monocytes collected from wild-type mice, the CD11c/CD115 ratio was significantly decreased in the AS1842856-treated group, compared to the control group (DMSO-treated group) (FIG. 5).

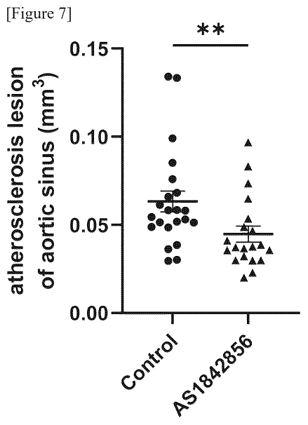

Next, after the FoxO1 inhibitor AS1842856 (20 mg/kg) was intraperitoneally administered to wild-type mice (Syk^{+/+}) for 8 weeks, atherosclerosis lesions in the aortic sinus were evaluated using HE staining. Atherosclerosis was suppressed in the AS1842856-treated group, compared to the control group (DMSO-treated group) (FIG. 6 and FIG. 7).

### INDUSTRIAL APPLICABILITY

The present invention can be used as an agent for inhibiting atherosclerosis.

1. (canceled)

2. (canceled)

3. A method for preventing or treating atherosclerosis, comprising administering to a subject in need thereof a FoXO1 inhibitor in an effective amount thereof.

4. (canceled)

5. The method for preventing or treating atherosclerosis according to claim 3, wherein the FoXO1 inhibitor is AS1842856 [5-amino-7-(cyclohexylamino)-1-ethyl-6-fluoro-4-oxo-1,4-dihydroquinoline-3-carboxylic acid].

6. The method for preventing or treating atherosclerosis according to claim 3, wherein the FoXO1 inhibitor is AS1708727 [2-Cyclopentyl-N-[2,4-dichloro-3-(isoquinolin-5-yloxymethyl)phenyl]N-methylacetamide].

7. The method for preventing or treating atherosclerosis according to claim 3, wherein the FoXO1 inhibitor is a siRNA.

8. The method for preventing or treating atherosclerosis according to claim 3, wherein the FoXO1 inhibitor is an anti-FoxO1 antibody.
